(19) United States
(12) Patent Application Publication
(10) Pub. No.: US 2010/0254923 A1
(43) Pub. Date: Oct. 7, 2010

(54) COMPOSITION AND PROCESS FOR PROTECTING CELLULAR TARGETS FROM AGING AND PHOTODAMAGE

(75) Inventors: Angelike GALDI, Westfield, NJ (US); Peter Foltis, Nutley, NJ (US); Anil Shah, East Windsor, NJ (US)

Correspondence Address: L'Oreal USA;Patent Department, 30 L'Oreal Way, Clark, NJ, 07066, US

(73) Assignee: L'ORÉAL, Paris (FR)

(21) Appl. No.: 12/754,192

(22) Filed: Apr. 5, 2010

Related U.S. Application Data
(60) Provisional application No. 61/166,978, filed on Apr. 6, 2009.

(51) Int. Cl. A61K 8/97; A61Q 17/04; A61K 31/216; A61P 17/00

(52) U.S. Cl. 424/60

(57) ABSTRACT

The present invention is directed to a composition and process for protecting cellular targets from UV rays and free radical-induced damage by applying onto skin a composition containing: (a) at least one antioxidant chosen from 4-hydroxybenzylidene malonate derivatives and 4-hydroxycinnamate derivatives; (b) at least one extract of the plant Cassia alata; and (c) at least one sunscreen active.

This application claims the benefit of priority under 35 U.S.C. §119(e) from U.S. provisional application Ser. No. 61/166,978, filed Apr. 6, 2009, incorporated herein by reference in its entirety.

### BACKGROUND OF THE INVENTION

Aging skin is the result of more than just chronological age. Skin is exposed to various environmental stresses, such as UV rays, which cause free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical which may attack adjacent fatty acids to generate new carbon radicals. This process can lead to a chain reaction producing lipid peroxidation products. Damage to the cell membrane can result in loss of cell permeability, increased intercellular ionic concentration and/or decreased ability to excrete or detoxify waste products. The end result is a loss of elasticity of the skin and the appearance of wrinkles leading to premature ageing of the skin. This process is commonly referred to as photo-aging.

Thus, it is an object of the present invention to provide a composition and process for protecting cellular targets from aging and photo-damage caused by UV light, in general, and free radicals formed thereby, in particular.

### BRIEF SUMMARY OF THE INVENTION

The present invention is directed to a composition for protecting cellular targets from UV rays and free radical damage containing:
(a) at least one antioxidant chosen from 4-hydroxybenzylidene malonate derivatives and 4-hydroxycinnamate derivatives such as those described in patent application WO 03/007 906;
(b) at least one extract of the plant Cassia alata; and
(c) at least one sunscreen active.

The present invention is also directed to a process for protecting cellular targets from UV rays and free radical-induced damage by topically applying the above-disclosed composition onto skin. The process for protecting includes both prophylactic and therapeutic inhibition of free radical-induced damage of the cellular targets.

### BRIEF DESCRIPTION OF THE DRAWINGS

FIG. 1 is a bar graph evidencing the photo protection efficacy of the present invention.

FIG. 2 is a bar graph evidencing the p53 efficacy of the present invention.

FIG. 3 is a bar graph evidencing the MMP-9 efficacy of the present invention.

### DETAILED DESCRIPTION OF THE INVENTION

Other than in the operating examples, and where otherwise indicated, all numbers expressing quantities of ingredients and/or reaction conditions are to be understood as being modified in all instances by the term “about”.

The present invention is based on the surprising and unexpected finding that the combination of at least one antioxidant chosen from 4-hydroxybenzylidene malonate derivative and a 4-hydroxycinnamate derivative, with extracts of the plant Cassia alata and at least one sunscreen active ingredient yields a composition that is highly effective at protecting cellular targets from free radicals caused by their exposure to UV rays.

Among the 4-hydroxybenzylidenemalonate derivatives and/or the 4-hydroxycinnamate derivatives that will preferentially be used are those corresponding to formula (I):

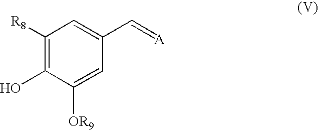

in which A is a chromophoric group that absorbs UV radiation, comprising two monovalent groups containing a carbonyl function;
R₈ denotes hydrogen, a linear or branched C₁-C₈ alkyl radical or a linear or branched C₁-C₈ alkoxy radical,
R₉ denotes a linear or branched C₁-C₈ alkyl radical.

Among these compounds, the ones that will be used more preferentially are those of formula (VIa) below:

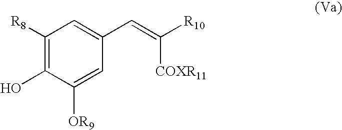

in which
R₈ denotes hydrogen, a linear or branched C₁-C₈ alkyl radical or a linear or branched C₁-C₈ alkoxy radical,
R₉ denotes hydrogen or a linear or branched C₁-C₈ alkyl radical,
R₁₀ is chosen from —C(O)CH₃, —CO₂R₁₂, —C(O)NH₂, and —C(O)N(R₁₃)₂,
X denotes O or NH,
R₁₁ denotes a linear or branched C₁-C₃₀ alkyl radical,
R₁₂ represents a linear or branched C₁-C₂₀ alkyl radical,
each R₁₃ independently represents a linear or branched C₁-C₈ alkyl radical.

Among these compounds, the ones that will be used more preferentially are those of formula (VIb) or (VIc) below:

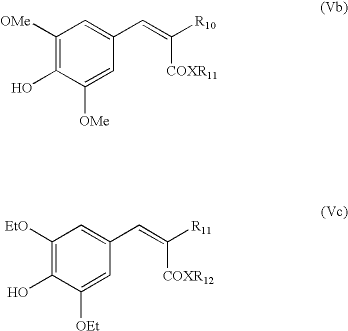

in which
R₁₀ denotes —CO₂R₁₂
R₁₁ denotes a linear or branched C₁-C₈ alkyl
R₁₂ denotes a linear or branched C₁-C₈ alkyl
X denotes O.

A particularly preferred compound for use in the present invention is diethylhexyl syringylidenemalonate (INCI name) having the following formula:

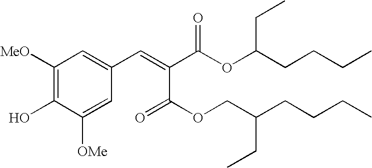

Which is commercially available from Merck KgaA under the trade name Oxynex® ST.

The 4-hydroxybenzylidenemalonate derivatives and/or the 4-hydroxycinnamate derivatives are typically employed in an amount of from about 0.1 to about 15% by weight, such as from about 0.1 to about 10% by weight, and from about 1 to about 5% by weight, based on the total weight of the composition.

The extracts used in accordance with the present invention are obtained from plants of the genus Caesalpiniaceae, more particularly from the relatively uncommon species Cassia alata, such as those described in US 2003/0180231, the entire content of which is hereby incorporated by reference.

Particularly preferred extracts of the plant Cassia alata are those commercially available from Cognis under the tradename DN-Age®.

The extracts are typically employed in an amount of from about 0.01 to about 10% by weight, such as from about 0.01 to about 5% by weight, and from about 0.04 to about 3% by weight, based on the total weight of the composition.

The composition also contains at least one sunscreen active ingredient chosen from UVA-active and/or UVB-active organic or mineral photoprotective agents that are water-soluble or liposoluble or insoluble in the cosmetic solvents commonly used.

Examples thereof include, but are not limited to, anthranilates; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; benzoxazole derivatives such as those described in patent applications EP 0 832 642; EP 1 027 883, EP 1 300 137 and DE 101 62 844; screening polymers and screening silicones such as those described especially in patent application WO 93/04665; dimers derived from α-alkylstyrene, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981, and mixtures thereof.

As examples of organic photoprotective agents, mention may be made of those denoted hereinbelow under their INCI name:

para-Aminobenzoic acid derivatives:

### PARA,

### Ethyl PABA,

Ethyl dihydroxypropyl PABA,
Ethylhexyl dimethyl PABA sold in particular under the name

### “Escalol 507” by ISP,

### Glyceryl PABA,

PEG-25 PABA sold under the name “Uvinul P25” by BASF.

Salicylic derivatives:

Homosalate sold under the name “Eusolex HMS” by Rona/EM Industries,
Ethylhexyl salicylate sold under the name “Neo Heliopan OS” by Haarmann and Reimer,
Dipropylene glycol salicylate sold under the name “Dipsal” by Scher,
TEA salicylate sold under the name “Neo Heliopan TS” by Haarmann and Reimer.

β,β-Diphenylacrylate derivatives:

Octocrylene sold in particular under the trade name “Uvinul N539” by BASF,
Etocrylene sold in particular under the trade name “Uvinul N35” by BASF.

Benzophenone derivatives:

Benzophenone-1 sold under the trade name “Uvinul 400” by BASF,
Benzophenone-2 sold under the trade name “Uvinul D50” by BASF,
Benzophenone-3 or Oxybenzone sold under the trade name “Uvinul M40” by BASF,
Benzophenone-4 sold under the trade name “Uvinul MS40” by BASF,

### Benzophenone-5,

Benzophenone-6 sold under the trade name “Helisorb 11” by Norquay,
Benzophenone-8 sold under the trade name “Spectra-Sorb UV-24” by American Cyanamid,
Benzophenone-9 sold under the trade name “Uvinul DS-49” by BASF,

### Benzophenone-12

Diethylaminohydroxybenzoylhexyl benzoate sold under the trade name “Uvinul A Plus” by BASF,

Benzylidenecamphor derivatives:

3-Benzylidenecamphor manufactured under the name “Mexoryl SD” by Chimex,
4-Methylbenzylidenecamphor sold under the name “Eusolex 6300” by Merck,
Benzylidenecamphorsulfonic acid manufactured under the name “Mexoryl SL” by Chimex,
Camphor benzalkonium methosulfate manufactured under the name “Mexoryl SO” by Chimex,
Terephthalylidenedicamphorsulfonic acid manufactured under the name “Mexoryl SX” by Chimex,
Polyacrylamidomethylbenzylidenecamphor manufactured under the name “Mexoryl SW” by Chimex.

Phenylbenzimidazole derivatives:

Phenylbenzimidazolesulfonic acid sold in particular under the trade name “Eusolex 232” by Merck,
Disodium phenyl dibenzimidazole tetrasulfonate sold under the trade name “Neo Heliopan AP” by Haarmann and Reimer.

Phenylbenzotriazole derivatives:

Drometrizole trisiloxane sold under the name “Silatrizole” by Rhodia Chimie,
Methylenebis(benzotriazolyl)tetramethylbutylphenol sold in solid form under the trade name “MIXXIM BB/100” by Fairmount Chemical, or in micronized form as an aqueous dispersion under the trade name “Tinosorb M” by Ciba Specialty Chemicals.

Triazine derivatives:

Bis(ethylhexyloxyphenol)methoxyphenyl triazine sold under the trade name “Tinosorb S” by Ciba-Geigy,
Ethylhexyltriazone sold in particular under the trade name “Uvinul T150” by BASF,
Diethylhexylbutamidotriazone sold under the trade name “Uvasorb HEB” by Sigma 3V,
2,4,6-tris(diisobutyl 4′-aminobenzalmalonate)-s-triazine.

Anthranilic derivatives:

Menthyl anthranilate sold under the trade name “Neo Heliopan MA” by Haarmann and Reimer.

Imidazoline derivatives:

Ethylhexyldimethoxybenzylidenedioxoimidazoline propionate.

Benzalmalonate derivatives:

Polyorganosiloxane containing benzalmalonate functions, for instance Polysilicone-15, sold under the trade name “Parsol SLX” by Hoffmann LaRoche

4,4-Diarylbutadiene derivatives:

1,1-Dicarboxy(2,2′-dimethylpropyl)-4,4-diphenylbutadiene

Benzoxazole derivatives:

2,4-bis[5-(1-dimethylpropyl)benzoxazol-2-yl (4-phenyl)imino]-6-(2-ethylhexyl)imino-1,3,5-triazine sold under the name Uvasorb K2A by Sigma 3V and mixtures thereof.

Examples of mineral photoprotective agents are chosen from pigments and even more preferably nanopigments (mean size of the primary particles: generally between 5 nm and 100 nm and preferably between 10 nm and 50 nm) of treated or untreated metal oxides such as, for example, nanopigments of titanium oxide (amorphous or crystallized in rutile and/or anatase form), of iron oxide, of zinc oxide, of zirconium oxide or of cerium oxide.

The treated nanopigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal (titanium or aluminium) alkoxides, poly-ethylene, silicones, proteins (collagen or elastin), alkanolamines, silicon oxides, metal oxides, sodium hexametaphosphate, alumina or glycerol.

The treated nanopigments may more particularly be, titanium oxides treated with:

silica and alumina, such as the products “Microtitanium Dioxide MT 500 SA” and “Microtitanium Dioxide MT 100 SA” from the company Tayca, and the products “Tioveil Fin”, “Tioveil OP”, “Tioveil MOTG” and “Tioveil IPM” from the company Tioxide,

alumina and aluminium stearate, such as the product “Microtitanium Dioxide MT 100 T” from the company Tayca,

alumina and aluminium laurate, such as the product “Microtitanium Dioxide MT 100 S” from the company Tayca,

iron oxides and iron stearate, such as the product “Microtitanium Dioxide MT 100 F” from the company Tayca,

silica, alumina and silicone, such as the products “Microtitanium Dioxide MT 100 SAS”, “Microtitanium Dioxide MT 600 SAS” and “Microtitanium Dioxide MT 500 SAS” from the company Tayca,

sodium hexametaphosphate, such as the product “Microtitanium Dioxide MT 150 W” from the company Tayca,

octyltrimethoxysilane, such as the product “T-805” from the company Degussa,

alumina and stearic acid, such as the product “UVT-M160” from the company Kemira,

alumina and glycerol, such as the product “UVT-M212” from the company Kemira,

alumina and silicone, such as the product “UVT-M262” from the company Kemira.

Other titanium oxide nanopigments treated with a silicone are preferably TiO₂ treated with octyltrimethylsilane and for which the mean size of the elementary particles is between 25 and 40 nm, such as the product sold under the trade name “T805” by the company Degussa Silices, TiO₂ treated with a polydimethylsiloxane and for which the mean size of the elementary particles is 21 nm, such as the product sold under the trade name “70250 Cardre UF TiO2SI3” by the company Cardre, anatase/rutile TiO₂ treated with a polydimethylhydrogenosiloxane and for which the mean size of the elementary particles is 25 nm, such as the product sold under the trade name “Microtitanium Dioxide USP Grade Hydrophobic” by the company Color Techniques.

The uncoated titanium oxide nanopigments are sold, for example, by the company Tayca under the trade names “Microtitanium Dioxide MT 500 B” or “Microtitanium Dioxide MT 600 B”, by the company Degussa under the name “P 25”, by the company Wackher under the name “Oxyde de titane transparent PW”, by the company Myoshi Kasei under the name “UFTR”, by the company Tomen under the name ITS and by the company Tioxide under the name “Tioveil AQ”.

The uncoated zinc oxide nanopigments are, for example:

those sold under the name “Z-Cote” by the company Sunsmart;

those sold under the name “Nanox” by the company Elementis;

those sold under the name “Nanogard WCD 2025” by the company Nanophase Technologies.

The coated zinc oxide nanopigments are, for example:

those sold under the name “Zinc Oxide CS-5” by the company Toshibi (ZnO coated with polymethylhydrogenosiloxane);

those sold under the name “Nanogard Zinc Oxide FN” by the company Nanophase Technologies (as a 40% dispersion in Finsolv TN, C₁₂-C₁₅ alkyl benzoate);

those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane);

those sold under the name “NFD Ultrafine ZNO” by the company Daikin (ZnO coated with perfluoroalkyl phosphate and copolymer based on perfluoroalkylethyl as a dispersion in cyclopentasiloxane);

those sold under the name “SPD-Z1” by the company Shin-Etsu (ZnO coated with silicone-grafted acrylic polymer, dispersed in cyclodimethylsiloxane);

those sold under the name “Escalol Z100” by the company ISP (alumina-treated ZnO dispersed in an ethylhexyl methoxycinnamate/PVP-hexadecene/methicone copolymer mixture);

those sold under the name “Fuji ZNO-SMS-10” by the company Fuji Pigment (ZnO coated with silica and polymethylsilsesquioxane);

those sold under the name “Nanox Gel TN” by the company Elementis (ZnO dispersed at a concentration of 55% in C₁₂-C₁₅ alkyl benzoate with hydroxystearic acid polycondensate).

The uncoated cerium oxide nanopigments are sold under the name “Colloidal Cerium Oxide” by the company Rhone-Poulenc.

The uncoated iron oxide nanopigments are sold, for example, by the company Arnaud under the names “Nanogard WCD 2002 (FE 45B)”, “Nanogard Iron FE 45 BL AQ”, “Nanogard FE 45R AQ” and “Nanogard WCD 2006 (FE 45R)” or by the company Mitsubishi under the name “TY-220”,

The coated iron oxide nanopigments are sold, for example, by the company Arnaud under the names “Nanogard WCD 2008 (FE 45B FN)”, “Nanogard WCD 2009 (FE 45B 556)”, “Nanogard FE 45 BL 345” and “Nanogard FE 45 BL” or by the company BASF under the name “Transparent Iron Oxide”.

Mention may also be made of mixtures of metal oxides, especially of titanium dioxide and of cerium dioxide, including the silica-coated equal-weight mixture of titanium dioxide and of cerium dioxide, sold by the company Ikeda under the name “Sunveil A”, and also the alumina, silica and silicone-coated mixture of titanium dioxide and of zinc dioxide, such as the product “M 261” sold by the company Kemira, or the alumina, silica and glycerol-coated mixture, of titanium dioxide and of zinc dioxide, such as the product “M 211” sold by the company Kemira.

The nanopigments may be introduced into the compositions according to the invention in unmodified form or in the form of pigmentary paste, i.e. as a mixture with a dispersant, as described, for example, in document GB-A-2 206 339.

The at least one sunscreen active is typically present in an amount of from about 0.1% to about 40% by weight, such as from about 1% to about 35% by weight, such as from about 1 to about 15% by weight, relative to the total weight of the composition.

The composition of the present invention may also contain agents for artificially tanning and/or browning the skin (self-tanning agents) and more particularly dihydroxyacetone (DHA). They are preferably present in amounts ranging from 0.1% to 10% by weight relative to the total weight of the composition.

The composition may also comprise standard cosmetic adjuvants chosen especially from fatty substances, organic solvents, ionic or nonionic, hydrophilic or lipophilic thickeners, softeners, humectants, opacifiers, stabilizers, emollients, silicones, antifoams, fragrances, preserving agents, anionic, cationic, nonionic, zwitterionic or amphoteric surfactants, active agents, fillers, polymers, propellants, acidifying or basifying agents or any other ingredient usually used in cosmetics and/or dermatology.

The fatty substances may consist of an oil or a wax or mixtures thereof. The term “oil” means a compound that is liquid at room temperature. The term “wax” means a compound that is solid or substantially solid at room temperature and whose melting point is generally greater than 35° C.

Oils that may be mentioned include mineral oils (paraffin); plant oils (sweet almond oil, macadamia oil, grapeseed oil or jojoba oil); synthetic oils, for instance perhydrosqualene, fatty alcohols, fatty acids or fatty esters (for instance the C₁₂-C₁₅ alkyl benzoate sold under the trade name “Finsolv TN” of “Witconol TN” by the company Witco, octyl palmitate, isopropyl lanolate and triglycerides, including capric/caprylic acid triglycerides), oxyethylenated or oxypropylenated fatty esters and ethers; silicone Oils (cyclomethicone and polydimethylsiloxanes, or PDMS) or fluoro oils, and polyalkylenes.

Waxy compounds that may be mentioned include paraffin, carnauba wax, beeswax and hydrogenated castor oil.

Among the organic solvents that may be mentioned are lower alcohols and polyols. These polyols may be chosen from glycols and glycol ethers, for instance ethylene glycol, propylene glycol, butylene glycol, dipropylene glycol or diethylene glycol.

Hydrophilic thickeners that may be mentioned include carboxyvinyl polymers such as the Carbopol products (carbomers) and the Pemulen products (acrylate/C10-C30-alkylacrylate copolymer); polyacrylamides, for instance the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name “Hostacerin AMPS” (CTFA name: ammonium polyacryldimethyltauramide); cellulose-based derivatives such as hydroxyethylcellulose; polysaccharides and especially gums such as xanthan gum; and mixtures thereof.

Lipophilic thickeners that may be mentioned include modified clays such as hectorite and its derivatives, for instance the products sold under the name bentone.

Among the active agents that may be mentioned are:

antipollution agents and/or free-radical scavengers;

depigmenting agents and/or propigmenting agents;

antiglycation agents;

NO-synthase inhibitors;

agents for stimulating the synthesis of dermal or epidermal macromolecules and/or for preventing their degradation;

agents for stimulating fibroblast proliferation; agents for stimulating keratinocyte proliferation;

muscle relaxants;

tensioning agents;

desquamating agents;

moisturizers;

anti-inflammatory agents;

agents acting on the energy metabolism of cells;

insect repellants;

substance P or CGRP antagonists.

Needless to say, a person skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be prepared according to techniques that are well known to those skilled in the art, in particular those intended for the preparation of emulsions of oil-in-water or water-in-oil type. They may be in particular in the form of a simple or complex emulsion (O/W, W/O, O/W/O or W/O/W emulsion) such as a cream or a milk, in the form of a gel or a cream-gel, or in the form of a lotion, a powder or a solid tube, and may optionally be packaged as an aerosol and may be in the form of a mousse or a spray.

The compositions according to the invention are preferably in the form of an oil-in-water or water-in-oil emulsion.

The emulsions generally contain at least one emulsifier chosen from amphoteric, anionic, cationic and nonionic emulsifiers, which are used alone or as a mixture.

The emulsifiers are appropriately chosen according to the emulsion to be obtained (W/O or O/W).

As emulsifying surfactants that may be used for the preparation of the W/O emulsions, examples that may be mentioned include sorbitan, glycerol or sugar alkyl esters or ethers; silicone surfactants, for instance dimethicone copolyols, such as the mixture of cyclomethicone and of dimethicone copolyol, sold under the name “DC 5225 C” by the company Dow Corning, and alkyldimethicone copolyols such as laurylmethicone copolyol sold under the name “Dow Corning 5200 Formulation Aid” by the company Dow Corning; cetyldimethicone copolyol, such as the product sold under the name Abil EM 90R by the company Goldschmidt, and the mixture of cetyldimethicone copolyol, of polyglyceryl isostearate (4 mol) and of hexyl laurate, sold under the name Abil WE O9 by the company Goldschmidt. One or more co-emulsifiers may also be added thereto, which may be chosen advantageously from the group comprising polyol alkyl esters. Polyol alkyl esters that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate; fatty alkyl ethers of sugars, especially polyalkylglucosides (APG) such as decylglucoside and laurylglucoside sold, for example, by the company Henkel under the respective names Plantaren 2000 and Plantaren 1200, cetostearyl glucoside optionally as a mixture with cetostearyl alcohol, sold, for example, under the name Montanov 68 by the company SEPPIC, under the name Tegocare CG90 by the company Goldschmidt and under the name Emulgade KE3302 by the company Henkel, and also arachidyl glucoside, for example in the form of a mixture of arachidyl alcohol, behenyl alcohol and arachidyl glucoside, sold under the name Montanov 202 by the company SEPPIC. According to one particular embodiment of the invention, the mixture of the alkylpolyglucoside as defined above with the corresponding fatty alcohol may be in the form of a self-emulsifying composition as described, for example, in document WO-A-92/06778.

When it is an emulsion, the aqueous phase of this emulsion may comprise a nonionic vesicular dispersion prepared according to known processes (Bangham, Standish and Watkins, J. Mol. Biol. 13, 238 (1965), FR 2 315 991 and FR 2 416 008).

The compositions according to the invention find their application in a large number of treatments, especially cosmetic treatments, of the skin, the lips and the hair, including the scalp, especially for protecting and/or caring for the skin, the lips and/or the hair, and/or for making up the skin and/or the lips.

The compositions, in an embodiment of the present invention, can employ at least one aesthetic modifier. Aesthetic modifiers can be used to enhance the texture of the composition. For example, the compositions of the current invention can have a matte, powdery, non greasy, soft texture. Aesthetic modifiers can be chosen from: Nylon-12, polymethylsilsesquioxane, styrene/acrylates copolymer, silica and/or mixtures thereof.

The present invention will be better understood from the examples that follow, all of which are intended for illustrative purposes only and are not meant to unduly limit the scope of the invention in any way.

### EXAMPLES

### Inventive Example

| Ingredients | % wt/wt |
|---|---|
| Sunscreen actives | 34.00 |
| Water | 18.0600 |
| Disodium EDTA | 0.100 |
| Sodium Chloride | 0.500 |
| Preservative | 8.550 |
| Emulsifier | 3.620 |
| Dicaprylyl Carbonate | 1.000 |
| Dicaprylyl Ether | 4.000 |
| Diethylhexyl | 3.000 |
| Syringylidenemalonate | |
| Thickener | 1.000 |
| Cyclomethicones | QS |
| Dimethicone | 2.500 |
| Aesthetic modifiers | 12.000 |
| Cassia Alata Leaf Extract (and) | 0.170 |
| Maltodextrin | |

### Placebo Example

| | % |
|---|---|
| Ingredients | wt/wt |
| Water | 18.060 |
| Disodium EDTA | 0.100 |
| Sodium Chloride | 0.500 |
| Preservative | 8.550 |
| Emulsifier | 3.620 |
| Dicaprylyl Carbonate | 1.000 |
| Dicaprylyl Ether | 4.000 |
| Thickener | 1.000 |
| Cyclomethicones | QS |
| Dimethicone | 2.500 |
| Aesthetic Modifiers | 12.000 |

Protection Factor/Erythema

Erythema is one of the acute inflammatory effects of UV exposure. This biomarker is a measure of protection and can be evaluated both visually and colorimetrically. Erythema is the biomarker visually evaluated in SPF determination for sunscreen efficacy. Topically applied anti-oxidants can also suppress erythema following UV exposure.

p53

The cellular protein, p53 is of paramount importance in the protection of cells from DNA damage. p53 is upregulated in response to UV exposure and binds to DNA to either regulate repair mechanisms or initiate apoptosis DNA repair mechanisms are initiated when the damage is minimal. With overwhelming DNA damage, p53 initiates apoptosis, which prevents the survival of damaged cells that would otherwise propagate with mutations. p53 is the most commonly mutated gene found in human cancers, particularly non-melanoma skin cancers.

MMP-9

MMP's (matrix metalloproteinases) are enzymes that breakdown specific collagens in the extracellular matrix. Normally, they provide a homeostatis for the dermis. However, MMP's, in particular, MMP-1, MMP-2 and MMP-9 are upregulated in response to UV exposure. In this study, MMP-9 levels were evaluated.

Protocol

Ten (10) healthy volunteers (between 18-60 yrs) with Fitzpatrick skin type I-ITT were recruited for this study. Five (5) sites (5 cm×7.5 cm) were marked on the back of each volunteer as follows:
Formula with anti-oxidants
Formula without anti-oxidants (Placebo)
Untreated/Exposed
Untreated/Unexposed (Naïve)

Each formula was applied at a rate of 2 μL/cm² to their corresponding marked areas on the back; no product was applied to either the Untreated/Exposed or the Untreated/Unexposed (Naïve) sites. Product applications were done daily for four (4) consecutive days. On Day 3, the individual minimal erythemal dose (MEDi) was determined in each individual on a separate area on the back using a range of UVR intensities (20-70 nJ/cm2 at 10 mJ intervals). On Day 4, the subjects received a single UVR dose of 5× the previously determined MEDi to each of the four sites. On Day 5, colorimetry readings and 4 mm punch biopsies were obtained from each site. The biopsies were processed for p53 and MMP-9.

Colorimetry (Protection Factor)

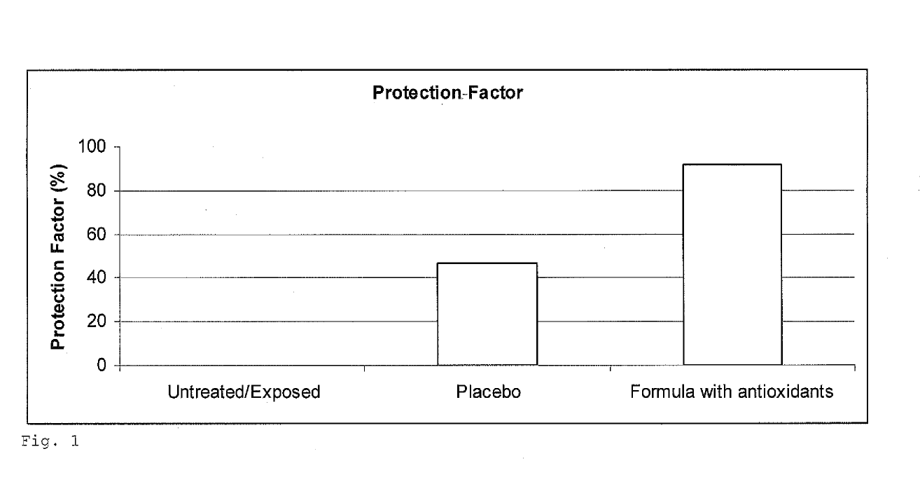

As can be seen in FIG. 1, the inventive formula provided a superior degree of photo protection which was determined by the degree of erythema (91.56%) measured on the skin using a colorimeter, as compared to the placebo.

p53

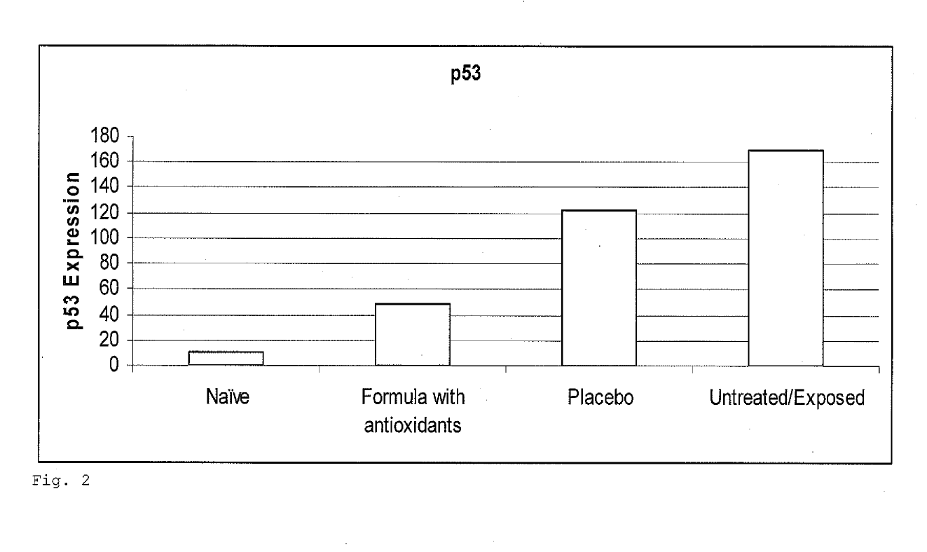

As can be seen in FIG. 2, the site treated with the inventive formula had significantly lower p53 expression than the Placebo sites. The Untreated/Exposed site was statistically higher in UV-induced p53 expression than all other sites.

MMP-9

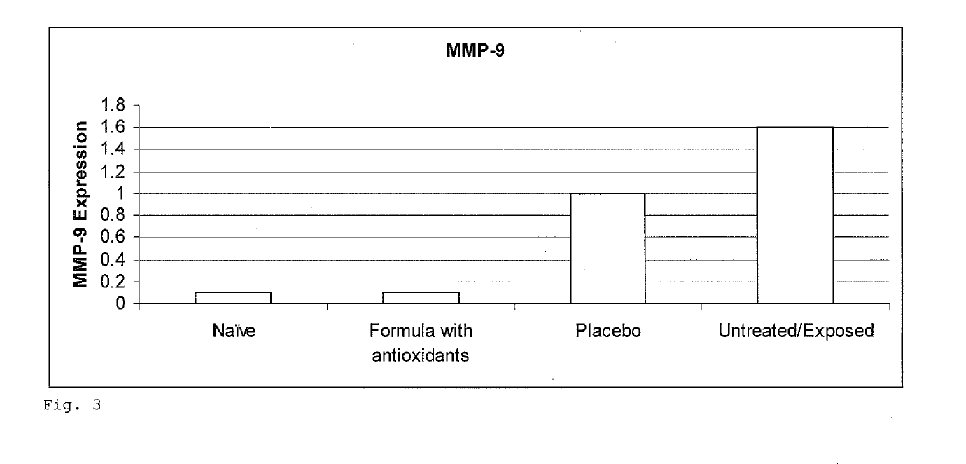

As can be seen in FIG. 3, the inventive formula with antioxidants was effective in suppressing MMP-9 expression following UV exposure. The expression was at parity with the expression on the Naïve site. The formula with antioxidants was more effective in suppressing MMP-9 expression than the Placebo. The Untreated/Exposed site was significantly higher than all other sites in MMP-9 expression.

What is claimed is:

1. A composition for protecting cellular targets from UV rays and free radical damage comprising:
(a) at least one antioxidant chosen from 4-hydroxybenzylidene malonate derivatives and 4-hydroxycinnamate derivatives;
(b) at least one extract of the plant Cassia alata; and
(c) at least one sunscreen active.

2. The composition of claim 1 wherein (a) is diethylhexyl syringylidenemalonate.

3. The composition of claim 1 wherein (a) is present in an amount of from about 0.1 to about 15% by weight, based on the total weight of the composition.

4. The composition of claim 1 wherein (a) is present in an amount of from about 1 to about 5% by weight, based on the total weight of the composition.

5. The composition of claim 1 wherein (b) is present in an amount of from about 0.01 to about 10% by weight, based on the total weight of the composition.

6. The composition of claim 1 wherein (b) is present in an amount of from about 0.04 to about 3% by weight, based on the total weight of the composition.

7. The composition of claim 1 wherein (c) is present in an amount of from about 0.1 to about 40% by weight, based on the total weight of the composition.

8. The composition of claim 1 wherein (c) is present in an amount of from about 1 to about 25% by weight, based on the total weight of the composition.

9. The composition of claim 1, further comprising an aesthetic modifier.

10. The composition of claim 9, wherein the aesthetic modifier is selected from Nylon-12,
polymethylsilsesquioxane, styrene/acrylates copolymer, silica and/or mixtures thereof.

11. A process for protecting cellular targets from free radical-induced damage comprising applying onto skin a composition containing:
(a) at least one antioxidant chosen from 4-hydroxybenzylidene malonate derivatives and 4-hydroxycinnamate derivatives;
(b) at least one extract of the plant Cassia alata; and
(c) at least one sunscreen active.

12. The process of claim 11 wherein (a) is diethylhexyl syringylidenemalonate.

13. The process of claim 11 wherein (a) is present in an amount of from about 0.1 to about 15% by weight, based on the total weight of the composition.

14. The process of claim 11 wherein (a) is present in an amount of from about 1 to about 5% by weight, based on the total weight of the composition.

15. The process of claim 11 wherein (b) is present in an amount of from about 0.01 to about 10% by weight, based on the total weight of the composition.

16. The process of claim 11 wherein (b) is present in an amount of from about 0.04 to about 3% by weight, based on the total weight of the composition.

17. The process of claim 11 wherein (c) is present in an amount of from about 0.1 to about 40% by weight, based on the total weight of the composition.

18. The process of claim 11 wherein (c) is present in an amount of from about 1 to about 25% by weight, based on the total weight of the composition.

19. The process of claim 11, further comprising an aesthetic modifier.

20. The process of claim 19, wherein the aesthetic modifier is selected from Nylon-12, polymethylsilsesquioxane, styrene/acrylates copolymer, silica and/or mixtures thereof.
